(19) United States
(12) Patent Application Publication
(10) Pub. No.: US 2026/0216138 A1
(43) Pub. Date: Jul. 30, 2026

(54) IDENTIFICATION OF OTUD7B INHIBITOR 7BI AND ITS APPLICATION IN REDUCING GROWTH OF NSCLC AND LEUKEMIA CELLS

(71) Applicants: The University of North Carolina at Chapel Hill, Chapel Hill, NC (US); Atomwise Inc., San Francisco, CA (US)

(72) Inventors: Pengda Liu, Chapel Hill, NC (US); Jianfeng Chen, Chapel Hill, NC (US); Nicholas Brown, Chapel Hill, NC (US); Christian Laggner, San Mateo, CA (US)

(21) Appl. No.: 19/147,353

(22) PCT Filed: Jan. 11, 2024
(86) PCT No.: PCT/US2024/011232

§ 371 Date: Jul. 11, 2025

Related U.S. Application Data
(60) Provisional application No. 63/439,017, filed on Jan. 13, 2023.
(60) Provisional application No. 63/439,547, filed on Jan. 17, 2023.

(51) Int. Cl. A61K 31/4155; A61K 45/06; A61P 35/00; A61P 35/02; C07D 231/40

(52) U.S. Cl. CPC A61K 31/4155; A61K 45/06; A61P 35/00; A61P 35/02; C07D 231/40

(57) ABSTRACT

Provided is a composition for targeting OTUD7B. The composition includes a component sufficient to block or reduce OTUD7B-mediated deubiquitination of GβL in a cell. The component can include 7Bi and variants thereof. Also provided are methods of treating cancer and related conditions, including administering to a cancer patient a OTUD7B catalytic inhibitor, including a 7Bi or variant thereof.

### CROSS-REFERENCE TO RELATED APPLICATIONS

This application claims benefit of U.S. Provisional Patent Application Ser. No. 63/439,017, filed Jan. 13, 2023, and U.S. Provisional Patent Application Ser. No. 63/439,547, filed Jan. 17, 2023, each of which are herein incorporated by reference in their entirety.

### REFERENCE TO SEQUENCE LISTING XML

The sequence listing associated with the instant disclosure has been electronically submitted to the United States Patent and Trademark Office via the Patent Center as a 4,944 byte Unicode UTF-8 XML file created on Dec. 26, 2023 and entitled “4210.0536 Sequence Listing”. The sequence listing submitted via Patent Center is hereby incorporated by reference in its entirety.

### TECHNICAL FIELD

The presently disclosed subject matter is directed to identification of OTUD7B inhibitor 7Bi and its application in reducing growth of NSCLC and leukemia cells.

### BACKGROUND

Protein deubiquitinases are an important family of enzymes commonly deregulated in cancer. Cancer is a disease that impacts people of all ages and demographics. There is a need for new and improved treatments for cancer, including non-small cell lung cancer, leukemia and breast cancer.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided are compositions for targeting OTUD7B, the composition comprising a component sufficient to block and/or reduce OTUD7B-mediated deubiquitination of GβL in a cell, wherein the component comprises 7Bi and variants thereof. In some aspects, the component is a catalytic inhibitor, wherein the catalytic inhibitor inhibits the catalytic activity of OTUD7B. In some aspects, blocking and/or reducing OTUD7B-mediated deubiquitination of GβL substantially inhibits, reduces and/or alters Akt-pS473 signaling in a cell, optionally wherein the Akt-pS473 signaling in a cell is inhibited, reduced and/or altered by about 10% to about 90%, or about 20% to about 80%, or about 30% to about 70%, or about 50% or more. In some aspects, the cell is a human cell. In some aspects, the composition is configured for in vivo administration to a subject. In some aspects, 7Bi comprises the following structure:

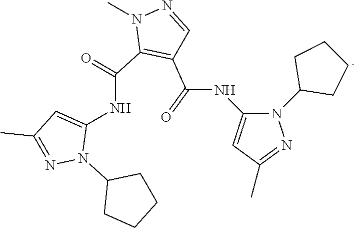 [SMILES: Cc1cc(NC(=O)c2cnn(C)c2C(=O)Nc2cc(C)nn2C2CCCC2)n(C2CCCC2)n1]

In some aspects, 7Bi is in the composition at a concentration sufficient to provide a dosage of about 10 μM when administered to a subject, optionally about 1 μM to about 20 μM. In some aspects, the composition comprises a delivery vehicle for the component, optionally wherein the delivery vehicle is an expression vector, nanoparticle, liposome or vesicle. In some aspects, the composition is configured for treating a cancer, optionally wherein the cancer is breast cancer, lung cancer, or leukemia.

In some embodiments, provided herein is an OTUD7B catalytic inhibitor, the catalytic inhibitor comprising a 7Bi compound or variant thereof. In some aspects, the catalytic inhibitor is configured to substantially limit cancer cell growth in vivo by blocking and/or reducing OTUD7B-mediated deubiquitination of GβL in a cell. In some aspects, the 7Bi compound comprises the following structure:

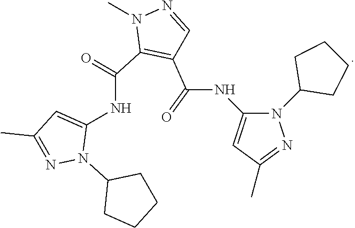 [SMILES: Cc1cc(NC(=O)c2cnn(C)c2C(=O)Nc2cc(C)nn2C2CCCC2)n(C2CCCC2)n1]

In some embodiments, provided herein are methods of treating cancer and related conditions, the methods comprising administering to a subject having cancer or suspected of suffering from cancer a composition comprising a OTUD7B catalytic inhibitor, optionally wherein the OTUD7B catalytic inhibitor comprises a 7Bi or variant thereof. In some aspects, the component blocks and or reduces OTUD7B-mediated deubiquitination of GPL in a cell in the subject. In some aspects, the component is a catalytic inhibitor, wherein the catalytic inhibitor inhibits the catalytic activity of OTUD7B. In some aspects, blocking and/or reducing OTUD7B-mediated deubiquitination of GβL substantially inhibits, reduces and/or alters Akt-pS473 signaling in a cell, optionally wherein the Akt-pS473 signaling in a cell is inhibited, reduced and/or altered by about 10% to about 90%, or about 20% to about 80%, or about 30% to about 70%, or about 50% or more. In some aspects, the 7Bi comprises the following structure:

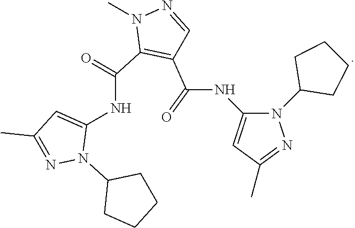 [SMILES: Cc1cc(NC(=O)c2cnn(C)c2C(=O)Nc2cc(C)nn2C2CCCC2)n(C2CCCC2)n1]

In some aspects, the subject is suffering from a cancer or is believed to be suffering from a cancer, optionally wherein the cancer is breast cancer, lung cancer, or leukemia. In some aspects, 7Bi is in the composition at a concentration sufficient to provide a dosage of about 10 μM when administered to a subject, optionally about 1 μM to about 20 M. In some aspects, the composition comprises a delivery vehicle for the component, optionally wherein the delivery vehicle is an expression vector, nanoparticle, liposome or vesicle. In some aspects, the composition is co-administered to the subject with at least one chemotherapeutic drug.

In some embodiments, provided are methods of blocking and/or reducing OTUD7B-mediated deubiquitination of GβL in a cell, the method comprising administering to a cell a composition comprising a component that targets OTUD7B in the cell, wherein the component comprises 7Bi or variant thereof.

These and other objects are achieved in whole or in part by the presently disclosed subject matter. Other objects and advantages of the presently disclosed subject matter will become apparent to those skilled in the art after a study of the following description, Drawings and Examples.

### BRIEF DESCRIPTION OF THE FIGURES

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following figures.

FIGS. 1A-1I show results of experiments searching for OTUD7B small molecule inhibitors. (A) A cartoon illustration for the pipeline of searching for OTUD7B small molecule inhibitors. The AtomNet technology was used to search a 4-million small molecule library for hits fitting into the OTUD7B-OTU structure. Top hits were purchased and effects of OTUD7B inhibition by these small molecules were tested by both Akt-pS473 ELISA and western blotting in A549 cells. Overlapping hits from two assays were identified and tested further. (B) Immuno-blot (IB) analyses of whole cell lysates (WCL) derived from control or OTUD7B depleted A549 cells. (C) Representative colony formation analyses images using A549 cells obtained in B and quantified in D. (E) A heatmap illustration of Akt-pS473 ELISA results obtained from treating A549 cells with 10 mM of each indicated chemical for 16 hrs. (F) A list of chemicals with an ability to suppress 30% Akt-pS473 signals from assays in E. (G) IB analyses of WCL from A549 cells treated with indicated compounds for 16 hrs. (H) A list of chemicals with an ability to suppress 10% Akt-pS473 signals from assays in G. (T) A list of overlapped hits from both F and H.

FIGS. 2A-2L show results of validation for OTUD7B small molecule inhibitors in cells. (A-D) IB analyses of WCL from indicated NSCLC cells treated with 10 mM of indicated compounds for 16 hrs. (E, F) IB analyses of WCL from either A549 (E) or HEK293 (F) cells treated with indicated doses of compound #19 for 16 hrs. (G) IB analyses of Ni-NTA pulldowns and WCL from HEK293 cells transfected with indicated DNA constructs. Where indicated, cells were treated with 10 mM of indicated compounds for 16 hrs before cell collection. (H-L) Quantifications of cell viability by MTT assays in A549 cells treated with indicated doses of indicated compounds for 72 hrs. Error bars were calculated as mean+/−SD, n=3. *p<0.05 (one-way ANOVA test).

FIGS. 3A-3Q show results of experiments evaluating whether 7Bi inhibits OTUD7B activity in vitro and suppress cell growth in NSCLC cells. (A-B) A representative gel image (A) for the in vitro OTUD7B deubiquitination assay incubating 5 nM of recombinant OTUD7B with 0.5 mM of K11-linked di-ub for 10 min in the presence of indicated doses of 7Bi. Mono-ub was quantified for IC50 value determination (B). (C-D) A representative gel image (C) for the in vitro USP21 deubiquitination assays incubating 80 nM of recombinant active USP21 proteins with 0.5 mM of K11-linked di-ub for 10 min in the presence of indicated doses of 7Bi. Mono-ub was quantified for IC50 value determination (D). (E-H) In vitro deubiquitination assays using ubiquitin-AMC as a substrate and indicated deubiquitinases. Normalized reading vs. reaction time periods were plotted. (I) A representative gel image for the in vitro OTUD7B deubiquitination assay incubating 5 nM of recombinant active OTUD7B proteins with 0.5 mM of K11-linked di-ub for 10 min in the presence of 100 mM of 7Bi (from either Mcule (90% purity) or recrystallized (99% purity)). (J) IB analysis of WCL derived from HEK293 cells treated with 10 mM of 7Bi (from either Mcule (90% purity) or HPLC purified (99% purity)). (K) IB analyses of GbL-IP and WCL from A549 cells treated with control or 10 mM 7Bi for 16 hrs. (L) IB analyses of HA-IP and WCL from HEK293 cells transfected with indicated DNA constructs. Where indicated, indicated doses of 7Bi were used to treat cells for 16 hrs before cell collection. (M) Representative colony formation images from indicated NSCLC cells treated with indicated doses of 7Bi once for 10 days and quantified in N-P. Error bars were calculated as mean+/−SD, n=3. *p<0.05 (one-way ANOVA test). (Q) Quantifications of cell viability by MTT assays in HEK293 cells treated with indicated doses of indicated compounds for 72 hrs. Error bars were calculated as mean+/−SD, n=3. *p<0.05 (one-way ANOVA test).

FIGS. 4A-4N show results of experiments evaluating OTUD7B inactivation suppresses leukemia cell proliferation. (A-C) IB analyses of WCL from indicated cells with depletion of OTUD7B by shRNAs. (D) IB analyses of GbL-IP and WCL from indicated K562 cells. (E-G) Quantifications of cell viability by MTT assays in indicated cells treated with indicated doses of indicated compounds for 72 hrs. Error bars were calculated as mean+/−SD, n=3. *p<0.05 (one-way ANOVA test). (H-J) IB analyses of WCL from indicated cells treated with indicated doses of 7Bi for 16 hrs. (H) IB analyses of GbL-IP and WCL from K562 cells treated with control or 10 mM 7Bi for 16 hrs. (L-N) Quantifications of cell viability by MTT assays in indicated cells treated with indicated doses of indicated compounds for 72 hrs. Error bars were calculated as mean+/−SD, n=3. *p<0.05 (one-way ANOVA test).

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand”, “coding sequence” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Also encompassed are any and all nucleotide sequences that encode the disclosed amino acid sequences, including but not limited to those disclosed in the corresponding GENBANK® entries.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

The terms “modulate” or “alter” are used interchangeably and refer to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the terms “modulate” and/or “alter” can mean “inhibit” or “suppress”, but the use of the words “modulate” and/or “alter” are not limited to this definition.

As used herein, the terms “inhibit”, “suppress”, “repress”, “downregulate”, “loss of function”, “block of function”, and grammatical variants thereof are used interchangeably and refer to an activity whereby gene expression (e.g., a level of an RNA encoding one or more gene products) is reduced below that observed in the absence of a composition of the presently disclosed subject matter. In some embodiments, inhibition results in a decrease in the steady state level of a target RNA.

The term “RNA” refers to a molecule comprising at least one ribonucleotide residue. By “ribonucleotide” is meant a nucleotide with a hydroxyl group at the 2′ position of a D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

The term “transcription factor” generally refers to a protein that modulates gene expression, such as by interaction with the cis-regulatory element and/or cellular components for transcription, including RNA Polymerase, Transcription Associated Factors (TAFs), chromatin-remodeling proteins, reverse tet-responsive transcriptional activator, and any other relevant protein that impacts gene transcription.

The term “promoter” defines a region within a gene that is positioned 5′ to a coding region of a same gene and functions to direct transcription of the coding region. The promoter region includes a transcriptional start site and at least one cis-regulatory element. The term “promoter” also includes functional portions of a promoter region, wherein the functional portion is sufficient for gene transcription. To determine nucleotide sequences that are functional, the expression of a reporter gene is assayed when variably placed under the direction of a promoter region fragment.

The terms “active”, “functional” and “physiological”, as used for example in “enzymatically active”, “functional chromatin” and “physiologically accurate”, and variations thereof, refer to the states of genes, regulatory components, chromatin, etc. that are reflective of the dynamic states of each as they exists naturally, or in vivo, in contrast to static or non-active states of each. Measurements, detections or screenings based on the active, functional and/or physiologically relevant states of biological indicators can be useful in elucidating a mechanism, or defining a disease state or phenotype, as it occurs naturally. This is in contrast to measurements taken based on static concentrations or quantities of a biological indicator that are not reflective of level of activity or function thereof.

As used herein, the terms “antibody” and “antibodies” refer to proteins comprising one or more polypeptides substantially encoded by immunoglobulin genes or fragments of immunoglobulin genes. The presently disclosed subject matter also includes functional equivalents of the antibodies of the presently disclosed subject matter. As used herein, the phrase “functional equivalent” as it refers to an antibody refers to a molecule that has binding characteristics that are comparable to those of a given antibody. In some embodiments, chimerized, humanized, and single chain antibodies, as well as fragments thereof, are considered functional equivalents of the corresponding antibodies upon which they are based. In some embodiments, the presently disclosed subject matter provides methods for identifying, characterizing and/or developing disease-related components of a gene-specific chromatin regulatory protein complex, wherein one or more antibodies can be used directly, or in assays related thereto, in the identification, characterization and/or isolation of such components.

The term “substantially identical”, as used herein to describe a degree of similarity between nucleotide sequences, peptide sequences and/or amino acid sequences refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide identity, when compared and aligned for maximum correspondence, as measured using a sequence comparison algorithm or by visual inspection.

As used herein, the terms “detectable moiety”, “detectable label”, and “detectable agent” refer to any molecule that can be detected by any moiety that can be added to a chemoprobe, antigen, inhibitor, marker, reagent and/or antibody, or a fragment or derivative thereof, that allows for the detection of the chemoprobe, antigen, inhibitor, marker, reagent and/or antibody, fragment, or derivative in vitro and/or in vivo. Representative detectable moieties include, but are not limited to, chromophores, fluorescent moieties, radioacite labels, affinity probes, enzymes, antigens, groups with specific reactivity, chemiluminescent moieties, and electrochemically detectable moieties, etc. In some embodiments, the antibodies are biotinylated.

The term “prodrug” refers to a derivative of a compound described herein, the pharmacologic action of which results from the conversion by chemical or metabolic processes in vivo to the active compound. Prodrugs can include compounds wherein an amino acid residue, or a polypeptide chain of two or more (e.g., two, three or four) amino acid residues is covalently joined through an amide or ester bond to a free amino, hydroxyl or carboxylic acid group of a compound. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

“Alkyl” refers to a straight or branched hydrocarbon chain radical consisting solely of carbon and hydrogen atoms, containing no unsaturation, having from one to ten carbon atoms (e.g., (C1-10)alkyl or C1-10 alkyl). Whenever it appears herein, a numerical range such as “I to 10” refers to each integer in the given range—e.g., “I to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2 where each Ra is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C2-10)alkenyl or C2-10 alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including IO carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C2-10)alkynyl or C2-10 alkynyl). Whenever it appears herein, a numerical range such as “2 to IO” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C3-10)cycloalkyl or C3-10 cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively. The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, —N(Ra)C(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)-attached through the carbonyl carbon wherein the alkoxy group has the indicated number of carbon atoms. Thus a (C1-6)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O) wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl) C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is I or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is I or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)2-N(Ra)—C(═O)— radical, where Ra is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is I or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(Ra)2 radical group, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(Ra)2 group has two Ra substituents other than hydrogen, they can be combined with the nitrogen atom to form a 4-, 5-, 6- or 7-membered ring. For example, —N(Ra)2 is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHRa, and —NRaRa each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)2 or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R2 of —N(R)2 of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3rd Ed., John Wiley & Sons, New York, N.Y., 1999, which is incorporated herein by reference in its entirety.

“Aromatic” or “aryl” or “Ar” refers to an aromatic radical with six to ten ring atoms (e.g., C6-C10 aromatic or C6-C10 aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PO3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PO3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3rd Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively. “Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively. “Heteroaryl” or “heteroaromatic” or “HetAr” or “Het” refers to a 5- to 18-membered aromatic radical (e.g., Cs-Cn heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms. 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. AN-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9, I 0-hexahydrocycloocta[d]pyrimidinyl, 5,6,7,8,9, I 0-hexahydrocycloocta[d]pyridazinyl, 5,6,7,8,9, I 0-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9, I 0, I 0a octahydrobenzo[h]quinazolinyl, 1-phenyl-IH-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —ORa, —SRa, —OC(O)Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —ORa, —SRa, —OC(O) Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.
“Nitro” refers to the —NO2 radical.
“Oxa” refers to the —O— radical.
“Oxo” refers to the ═O radical.
“Isomers” are different compounds that have the same molecular formula.
“Stereoisomers” are isomers that differ only in the way the atoms are arranged in space—i.e., having a different stereochemical configuration.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other. The absolute stereochemistry is specified according to the Cahn Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line.

Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R) or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle' s reagents, or derivatization of a compounds using a chiral compound such as Mosher' s acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts: or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)—(optionally substituted alkyl), —S(O)—(optionally substituted amino), —S(O)—(optionally substituted aryl), —S(O)— (optionally substituted heteroaryl) and —S(O)—(optionally substituted heterocycloalkyl). [0097] “Sulfonyl” refers to groups that include —S(O2)—H, —S(O2)— (optionally substituted alkyl), —S(O2)-(optionally substituted amino), —S(O2)-(optionally substituted aryl), —S(O2)-(optionally substituted heteroaryl), and —S(O2)-(optionally substituted heterocycloalkyl). [0098]“Sulfonamidyl” or “sulfonamido” refers to a —S(═O)2-NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)2-NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.
“Sulfoxyl” refers to a —S(═O)2OH radical.
“Sulfonate” refers to a —S(═O)2-OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively. [00101] Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### Pharmaceutical Compositions

The disclosure provides a pharmaceutical composition for use in the treatment of the diseases and conditions described herein. In some embodiments, the disclosure provides a pharmaceutical composition comprising a therapeutically effective amount of a component sufficient to block and/or reduce OTUD7B-mediated deubiquitination of Akt-pS473 in a cell, wherein the component comprises 7Bi and variants thereof.

The pharmaceutical compositions are typically formulated to provide a therapeutically effective amount of 7Bi or variant thereof. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

In some embodiments, the concentration of 7Bi or variant thereof provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of 7Bi or variant thereof provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of 7Bi or variant thereof provided in the pharmaceutical compositions of the disclosure is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of 7Bi or variant thereof provided in the pharmaceutical compositions of the disclosure is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of 7Bi or variant thereof provided in the pharmaceutical compositions of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of any one of the pharmaceutical compositions of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the compounds provided according to the disclosure is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the disclosure provides a pharmaceutical composition for oral administration containing 7Bi or variant thereof and their features and limitations as described herein, or pharmaceutically acceptable analogs, derivatives, salts, solvates, hydrates, cocrystals, or prodrugs thereof, described herein, and a pharmaceutical excipient suitable for administration.

In preferred embodiments, the disclosure provides a solid pharmaceutical composition for oral administration containing 7Bi or variant thereof and their features and limitations as described herein, or pharmaceutically acceptable analogs, derivatives, salts, solvates, hydrates, cocrystals, or prodrugs thereof, and a pharmaceutical excipient suitable for administration. In some embodiments, the composition further contains an effective amount of an additional active pharmaceutical ingredient. Such additional active pharmaceutical ingredients may also include those compounds used for sensitizing cells to additional agent(s).

In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may beadded (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about I to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, asyloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-IO laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol. PEG-20 trioleate, PEG-40 sorbitan oleate. PEG-80 sorbitan laurate, polysorbate 20, polysorbate 80, POE-9 lauryl ether, POE-23 lauryl ether, POE-IO oleyl ether, POE-20 oleyl ether, POE-20 stearyl ether, tocopheryl PEG-I 00 succinate, PEG-24 cholesterol, polyglyceryl-10 oleate, Tween 40, Tween 60, sucrose monostearate, sucrose monolaurate, sucrose monopalmitate, PEG I0-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycolalkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides. [00163] In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, E-caprolactam, N-alkylpyrrolidone, N-hydroxyalky lpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon. caprolactone and isomers thereof, 8-valerolactone and isomers thereof, -butyrolactone and isomers thereof; and other solubilizers known in the art, such as dimethyl acetamide, dimethyl isosorbide, N-methyl pyrrolidones, monooctanoin, diethylene glycol monoethyl ether, and water. Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the disclosure provides a pharmaceutical composition for injection containing 7Bi or variant thereof and a pharmaceutical excipient suitable for injection. Components and amounts of compounds in the compositions areas described herein.

The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating 7Bi or variant thereof in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze drying techniques which yield a powder of the active ingredient plus any additional desired ingredient from a previously sterile-filtered solution thereof.

### Pharmaceutical Compositions for Topical Delivery

In preferred embodiments, the disclosure provides a pharmaceutical composition for transdermal delivery containing 7Bi or variant thereof and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants(e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of 7Bi or variant thereof in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

Pharmaceutical Compositions for Inhalation Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

The disclosure also provides kits. The kits include 7Bi or variant thereof in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient.

Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In a preferred embodiment, the kits are for use in the treatment of cancer or hyperproliferative disorders. In an embodiment, the kits described herein are for use in the treatment of EWS.

### Dosages and Dosing Regimens

The amounts of 7Bi or variant thereof administered, will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be morethan adequate, while in other cases still larger doses may be employed without causing any harmful side effect—e.g., by dividing such larger doses into several small doses for administration throughout the day. The dosage may be provided in units of mg/kg of body mass or in mg/m² of body surface area.

In some embodiments, dosing may be once, twice, three times, four times, five times, six times, or more than six times per day. Dosing may be once a month, once every two weeks, once a week, or once every other day. In some embodiments, 7Bi or variant thereof is administered for more than 1, 2, 3, 4, 5, 6, 7, 14, or 28 days. In some embodiments, 7Bi or variant thereof is administered for less than 28, 14, 7, 6, 5, 4, 3, 2, or 1 day. In yet another embodiment, the administration continues for more than about 6, 10, 14, 28 days, two months, six months, or one year. In some cases, continuous dosing is achieved and maintained as long as necessary.

In some embodiments, an effective dosage is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount may be administered in either single or multiple doses by any of the accepted modes of administration of agents having similar utilities, including rectal, buccal, intranasal and transdermal routes, by intra arterial injection, intravenously, intraperitoneally, parenterally, intramuscularly, subcutaneously, orally, topically, or as an inhalant.

### II. Subjects

The subject treated, screened, tested, or to which a dosage is administered, is desirably a human subject, although it is to be understood that the principles of the disclosed subject matter indicate that the compositions and methods are effective with respect to invertebrate and to all vertebrate species, including mammals, which are intended to be included in the term “subject”. Moreover, a mammal is understood to include any mammalian species in which screening is desirable, particularly agricultural and domestic mammalian species.

The disclosed methods and treatments are particularly useful in the treatment of warm-blooded vertebrates. Thus, the presently disclosed subject matter concerns mammals and birds.

### III. General Discussion

Protein ubiquitination plays critical roles in regulating protein function including protein stability, trafficking, binding partners, cellular localization, and others [1]. Protein ubiquitination is a result of a balanced action from both ubiquitination and deubiquitination, governed by E3 ubiquitin ligases and deubiquitinases (DUBs), respectively. There are about 100 deubiquitinases in mammals [2] with either cysteine protease or metalloprotease activities, largely divided into 7 families including USPs (ubiquitin-specific proteases), OTUs (ovarian tumor proteases), UCHs (ubiquitin C-terminal hydrolases), Josephins (machado-josephin domain proteases), MINDYs (MIU containing novel DUB family), JAMMs (Jab1/Mov34/Mpr1 Pad1 N-terminal proteases), and lastly ZUFSP (zinc finger with UFM1-specific peptidase) [3-5]. Among these DUB members, OTU family of DUBs have been tightly connected with both ubiquitin link-age specificity and cancer relevance. For example, multiple studies support an onco-genic role of OTUB1 in tumorigenesis. Specifically, OTUB1 prevents ER-associated degradation of PD-L1 to contribute to immune suppression [6], inhibits Ras mono-ubiquitination to trigger lung cancer development [7], promotes RohA activation to induce prostate cancer metastasis [8], stabilizes ATF6 to facilitate bladder cancer growth [9], deubiquitinates and stabilizes FOXM1 to promote renal cell carcinoma progression [10], interacts and stabilizes SLC7A11 to regulate ferroptosis [11], and stabilizes c-Myc to facilitate Myc-dependent multiple myeloma proliferation [12]. On the other hand, the roles of OTUD3 are cancer type dependent. Specifically, OTUD3 deubiquitinates and stabilizes PTEN [13] and p53 [14] to suppress breast tumorigenesis, but it stabilizes GRP78 to promote lung tumorigenesis [15]. In addition, OTUD3 differentially regulates nucleic acid sensing pathways—OTUD3 removes K63-linked ubiquitin moieties from MAVS to suppress MAVS activation in responding to cytosolic RNA challenges [16] while deubiquitinates K48-ubiqutinated cGAS to stabilize cGAS thus potentiating DNA sensing [17]. OTUD3 also deubiquitinates and stabilizes PPARδ to regulate energy metabolism and obesity [18].

In addition, an oncogenic function of OTUD7B in cancer has been well demonstrated. OTUD7B removes K63-linked ubiquitination on GPL to promote mTORC2 activation in facilitating NSCLC progression [19]. This effect is largely mediated by the major mTORC2 downstream target Akt. Akt is an oncogenic kinase hyperactivated in virtually all types of cancer [20]. mTORC2/GβL kinase complex phosphorylates Akt—Ser473 to promote full activation of Akt together with PDK1-mediated Akt-Thr308 phosphorylation. Thus, Akt-pS473 is a faithful marker for mTORC2 activation and OTUD7B-mediated GβL deubiquitination in cells. Notably, OTUD7B induced cleavage of K63-linked chains on GBL does not affect GβL's protein stability, but rather shifts GβL from mTORC1 to mTORC2, leading to subsequent mTORC2 activation [19]. antagonizes APC/C-mediated K11-linked substrate ubiquitination and proteolysis [21], deubiquitinates LSD1 to regulate its binding with co-REST and genomewide occupancy to fuel breast cancer metastasis [22], stabilizes ERα to promote breast cancer progression [23], and also regulates HIF-1a homeostasis in a proteasome-independent manner in renal cancer [24]. Notably, multiple ubiquitin linkages including K63 and K11, have been shown to be regulated by OTUD7B. In addition, OTUD7B participates in immune regulation. OTUD7B deubiquitinates and stabilizes TRAF3 to induce non-canonical NF-κB activation in immune regulation [25], deubiquitinates and stabilizes Zap70 in T cell activation [26], and removes K63-linked ubiquitin chains on p62 to enhance its oligomerization that promotes IRF3 degradation as a negative feedback mechanism to restrain IFN production [27]. Increased OTUD7B expression is observed in breast cancer [28] and lung cancer [29]. OTUD7B is a prognostic marker in diffuse large B-cell lymphoma [30] and NSCLC [31], as well as a potential therapeutic target for myocardial infarction by ameliorating fibrosis [32], and predicts poor responses to paclitaxel in TNBC [28]. Considering its oncogenic function in cancer and its great potential as a cancer drug target but without available inhibitors, here we aim to rely on the artificial intelligence technology, the AtomNet® model, to screen for small molecule OTUD7B inhibitors.

Thus, in some embodiments, provided herein is a putative small molecule OTUD7B inhibitor obtained from an AI-aided virtual screen of a 4-million compound library. We validated effects of the OTUD7B inhibitor (7Bi) in reducing Akt-pS473 signals in multiple NSCLC and HEK293 cells by blocking OTUD7B-governed GβL deubiquitination in cells, as well as inhibiting OTUD7B-mediated cleavage of K11-linked di-ub in an in vitro enzyme assay. Furthermore, we report in leukemia cells, either genetic depletion or 7Bi-mediated pharmacological inhibition of OTUD7B reduces Akt-pS473 via inhibiting the OTUD7B/GβL signaling axis. Together, our study identifies the first putative OTUD7B inhibitor showing activities both in cells and in vitro, with promising applications as a therapeutic agent in treating cancer with OTUD7B overexpression.

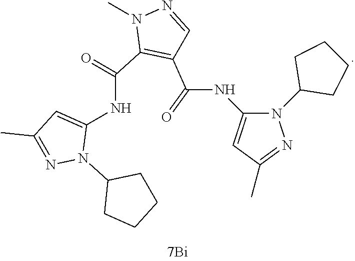 [SMILES: Cc1cc(NC(=O)c2cnn(C)c2C(=O)Nc2cc(C)nn2C2CCCC2)n(C2CCCC2)n1]

In some embodiments, provided are compositions for targeting OTUD7B, the composition comprising a component sufficient to block and/or reduce OTUD7B-mediated deubiquitination of GβL in a cell, wherein the component comprises 7Bi and variants thereof. In some embodiments, the component is a catalytic inhibitor, wherein the catalytic inhibitor inhibits the catalytic activity of OTUD7B. In some embodiments, blocking and/or reducing OTUD7B-mediated deubiquitination of GβL substantially inhibits, reduces and/or alters Akt-pS473 signaling in a cell, optionally wherein the Akt-pS473 signaling in a cell is inhibited, reduced and/or altered by about 10% to about 90%, or about 20% to about 80%, or about 30% to about 70%, or about 50% or more. In some embodiments, the cell is a human cell, optionally wherein the human cell is in vivo. In some embodiments, the composition is configured for in vivo administration to a subject. In some embodiments, 7Bi is in the composition at a concentration sufficient to provide a dosage of about 10 μM when administered to a subject, optionally about 1 μM to about 20 μM. In some embodiments, the composition comprises a delivery vehicle for the component, optionally wherein the delivery vehicle is an expression vector, nanoparticle, liposome or vesicle. In some embodiments, the composition is configured for treating a cancer, optionally wherein the cancer is breast cancer, lung cancer, or leukemia.

Also provided herein are OTUD7B catalytic inhibitors, the catalytic inhibitor comprising a 7Bi compound or variant thereof. In some embodiments, the catalytic inhibitor is configured to substantially limit cancer cell growth in vivo by blocking and/or reducing OTUD7B-mediated deubiquitination of GβL in a cell.

Also provided herein are methods of treating cancer and related conditions, the method comprising administering to a subject having cancer or suspected of suffering from cancer a composition comprising a OTUD7B catalytic inhibitor, optionally wherein the OTUD7B catalytic inhibitor comprises a 7Bi or variant thereof. In some embodiments, the component blocks and or reduces OTUD7B-mediated deubiquitination of GβL in a cell in the subject. In some embodiments, the component is a catalytic inhibitor, wherein the catalytic inhibitor inhibits the catalytic activity of OTUD7B. In some embodiments, blocking and/or reducing OTUD7B-mediated deubiquitination of GβL substantially inhibits, reduces and/or alters Akt-pS473 signaling in a cell, optionally wherein the Akt-pS473 signaling in a cell is inhibited, reduced and/or altered by about 10% to about 90%, or about 20% to about 80%, or about 30% to about 70%, or about 50% or more. In some embodiments, the subject is suffering from a cancer or is believed to be suffering from a cancer, optionally wherein the cancer is breast cancer, lung cancer, or leukemia. In some embodiments, 7Bi is in the composition at a concentration sufficient to provide a dosage of about 10 μM when administered to a subject, optionally about 1 μM to about 20 M. In some embodiments, the composition comprises a delivery vehicle for the component, optionally wherein the delivery vehicle is an expression vector, nanoparticle, liposome or vesicle. In some embodiments, the composition is co-administered to the subject with at least one chemotherapeutic drug.

Provided herein are methods of blocking and/or reducing OTUD7B-mediated deubiquitination of GβL in a cell, the method comprising administering to a cell a composition comprising a component that targets OTUD7B in the cell, wherein the component comprises 7Bi or variant thereof.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-6

### Cell Lines and Cell Culture

A549, H358, H520, H1299, HEK293T and HEK293 were cultured in DMEM medium supplemented with 10% FBS, 100 U penicillin and 100 mg/mL streptomycin. Acute myeloid leukemia cell lines K562, HL60 and leukemia monocytic cell line THP1 were cultured in RPMI 1640 medium supplemented with 10% FBS, 100 U penicillin and 100 mg/mL streptomycin. All the cell lines were cultured in 37° C. incubator with 5% CO2. HEK293 and HEK293T cells were purchased from UNC Lineberger Tissue Culture Facility. A549, H358, H520 and H1299 lung cancer cells were obtained from Dr. Chad Pecot lab at UNC. THP1 cells were obtained from Dr. Albert Baldwin lab at UNC. K562 and HL60 cells were obtained from Dr. Greg Wang lab at UNC.

### Antibodies

All antibodies were used at a 1:2000 dilution in TBST buffer with 5% non-fat milk and incubated at 4° C. with gentle shaking overnight for western blotting. Anti-HA anti-body (3724), anti-p-AKT (Ser473) (4060), anti-anti-p-p70 S6 Kinase (Thr389) (9234), anti-p-NDRG1 (Thr346) (89166), anti-p-GSK-3β (Ser9) (5558), anti-p-Fox01 (Thr24)/FoxO3a (Thr32) antibody (9464), anti-K63-linkage specific polyubiquitin anti-body (12930), anti-GβL antibody (3274), anti-Rictor antibody (2114), anti-Sin1 anti-body (12860), anti-mTOR antibody (2983), anti-AKT (4691), anti-rabbit IgG, HRP-linked antibody (7074) and anti-mouse IgG, HRP-linked antibody (7076) were obtained from Cell Signaling Technology. Anti-vinculin antibody (sc-25336) was obtained from Santa Cruz Biotechnology. Monoclonal anti-Flag antibody (F-3165, clone M2) and anti-α-tubulin antibody (T-5168) was obtained from Sigma. Anti-OTUD7B antibody (16605-1-AP) was obtained from Proteintech.

### Plasmids

HA-GβL, His-Ub and Flag-OTUD7B plasmids were constructed as described [19]. shRNA sequences used for OTUD7B depletion were as follows.

shOTUD7B-23:
(SEQ ID NO. 1)
GCAAGGAGGCTAAACAAAGTT
shOTUD7B-77:
(SEQ ID NO. 2)
CAAAGTTAAGCTCAACTAATT
shOTUD7B-95:
(SEQ ID NO. 3)
TGGAAATGCTCACGGTTTATA

### Immunoblot and Immunoprecipitations Analyses

Cells were harvested and lysed in EBC buffer (50 mM Tris pH 7.5, 120 mM NaCl, 0.5% NP-40) supplemented with protease inhibitor cocktail (EDTA-free, mini-tablet) (Bimake) and phosphatase inhibitor cocktail (Bimake) by rotating in the cold room for 10 min followed by high-speed centrifugation at 4° C. The protein concentrations in supernatants of whole cell lysates were measured by NanoDrop OneC using the Bio-Rad Bradford protein assay reagent. Equal amounts of whole cell lysates were resolved on SDS-PAGE and immunoblotted with indicated antibodies. For immunoprecipitation analysis, 1 mg of total cell lysate was incubated with the indicated beads for 3-4 hr at 4° C. The recovered immuno-complexes were washed thoroughly three times with NETN buffer (20 mM Tris, pH 8.0, 100 mM NaCl, 1 mM EDTA and 0.5% NP-40) before being resuspended in SDS sample buffer and resolved on SDS-PAGE and immunoblotted with indicated antibodies.

### Colony Formation Assays

500 cells from OTUD7B depleted A549 cells were seeded into each well of 6-well plates and cultured in the 37° C. incubator with 5% CO2 for 10-15 days until formation of visible colonies. Triplicates were performed. To test the effect of a given inhibitor on colony formation ability, 800 cells from A549, H520 and H1299 cell lines were seeded into each well of 6-well plates, respectively. Each inhibitor was added at indicated concentration the next day. Colonies were counted after 10 days of treatment. The colony staining method was as described [33]. Briefly, medium was removed, and colonies were fixed with fixation solution (20% acidic acid, 10% methanol) for at least 30 min and stained with 1.0% crystal violet overnight. Colonies were then washed several times with distilled water and air-dried. Colony numbers were counted manually. Three independent experiments were performed.

### Cell Viability and Proliferation Assay

2,000 cells from indicated NSLC cell lines were seeded into each well of 96-well plates. Cell viability was measured 3 days post-treatment using MTT assays following manufacturer's instruction. For cell viability assays with inhibitor treatment, 2,000 cells from indicated NSLC and HEK293 cell lines were seeded in 96-well plate. Inhibitor was added the next day at indicated concentration and cell viability was measured after 3-day treatment. For cell proliferation assays in THP1, K562 and HL60 cell lines, 1×106 cells from each indicated cell line were seeded in 12.5 cm2 cell culture flask and cells were counted by hemocytometer under microscope at indicated time to monitor cell proliferation. To test inhibitor effects on THP1, K562 and HL60 cell growth, 2×105 cells were seeded in 12.5 cm2 cell culture flask. Inhibitor was added the next day at indicated concentrations and cell number was counted after 3 days treatment. Three independent experiments were performed to generate error bars.

### Ubiquitin Chain Cleavage Assay

OTUD7B (residues 56-446), OTUD7A (residues 1-462) [34], USP21 (residues 196-565), and USP9X (residues 1551-1970) proteins were expressed in E. Coli BL21-Codon Plus (DE3)-RIL cells. Through an N-terminal GST tag, the DUBs were purified by affinity chromatography using glutathione sepharose 4B (GS4B) resin. Following the liberation of the DUB from the GST tag. the DUBs were subjected to size exclusion chromatography (SEC). To form the K11-linked di-ub, individual ubiquitins (K11R-substituted donor Ub and ΔGG acceptor ubiquitin) were purified and fluorescently-labeled on an N-terminal cysteine using either Cy3- (donor) or Cy5-maleimide (acceptor). The different ubiquitins were then added to a mixture containing UBA1, UBE2S, and MgATP to form di-ub. After an overnight incubation at room temperature, UBE2S was removed by cation-exchange chromatography and the di-ub was purified by SEC. These proteins were used to monitor DUB activity in the presence or absence of the various inhibitors. The inhibitors were incubated with DUBs in a reaction buffer (20 mM Hepes pH=8.0, 200 mM NaCl, 0.25 mg/mL BSA and 0.005% Tween-20) for 5 min at room temperature and then 0.5 uM fluorescently-labeled di-ub (K11-linked) was added to start the reaction. 5×SDS-PAGE loading buffer was used to quench the reaction at the indicated time point. Samples were run on SDS-PAGE and fluroescently scanned using Amersham™ Typhoon™ Biomolecular Imager. Mono-ubiquitin was quantified to generate an IC50 value. Three independent experiments were performed to generate the error bars.

### Ubiquitin-AMC Assay

50 μM 7Bi were incubated with 300 nM OTUD7B (residues 56-446), 10 μM OTUD7A (residues 1-462), 200 nM USP21 (residues 196-565), or 500 nM USP9X (residues 1551-1970) in reaction buffer (20 mM HEPES pH=8.0, 200 mM NaCl, 0.25 mg/mL BSA and 0.005% Tween-20) for 5 min at room temperature. Then, 2 uM ubiquitin-AMC was added to start the reaction. The fluorescence emission data was collected at an excitation wavelength of 355 nm and emission wavelength of 460 nm every 3 min. Reactions were completed three times to generate the error bars.

### Compound Purification

7Bi was purified by recrystallization from methanol and the purity is higher than 99%. The purity of this compound was determined by LC-MS. LC/MS was performed using an analytical instrument with the UV detector set to 220 nm, 254 nm, and 280 nm, and a single quadrupole mass spectrometer using electrospray ionization (ESI) source. Sample was injected (10 μL) onto a 4.6×50 mm, 1.8 μM, C18 column at room temperature. A linear gradient from 10% to 100% B (Acetonitrile+0.1% acetic Acid) in 6.0 min was followed by pumping 100% B for another 2 or 4 min with A being H2O+0.1% acetic acid. The flow rate was 1.0 mL/min.

### The AtomNet Model-Based Small Molecule Virtual Screen

The procedure is as described previously [35]. The virtual screen for new small molecule inhibitors of OTUD7B was performed using Atomwise's proprietary AI-based AtomNet® screening platform. We employed a single global AtomNet model to predict binding affinity of small molecules to a target protein. The catalytic OTU domain of OTUD7B has been crystallized in complex with a diubiquitin, monoubiquitin, and as an apo structure (PDB codes 5LRV, 5LRW, 5LRU, respectively [36]. As in our previous paper on OTUD7A, the area of the distal S1 ubiquitin-binding site was chosen due to the structural differences between OTUD7B and its close homolog OTUD7A to achieve potential ligand selectivity. The monoubiquitin-bound OTUD7B crystal structure 5LRW (after removal of the monoubiquitin), which had also served as the homology template for our previous work on OTUD7A, was chosen for the virtual screen, with the binding site located between residues W234, Q237, Q238, T239, Q241, N242, S245, G246, L247, Y249, W254, E257, W258, E260, L261, L264, L294, E295, E296, F297, H298, P330, F331, H390, and F391.

We again screened a filtered version of the Mcule small-molecule library (v20171018, 4,025,533 compounds). Compounds were docked and scored with the AtomNet model and ranked by their scores. After postprocessing, a set of 59 compounds containing diverse chemical scaffolds was selected from the top-scoring compounds and sourced from Mcule. Each compound was dissolved in DMSO with a concentration of 10 mM. All 10 mM DMSO stocks were analyzed by Mcule for purity via LC-MS. Only compounds that passed ≥85% purity were shipped and tested. The compound samples were assayed in a blinded way (chemical identities unknown to the lab researcher, with two negative control samples containing pure DMSO mixed in).

### Statistical Analysis

Statistical analysis was performed as described previously [35]. Briefly, SPSS 11.5 Statistical Software were used for statistical analyses. Asterisk (p≤0.05) was labeled for statistically significant. The results are shown as means±SD from independent experiments as indicated in figure legends. One-way ANOVA was used to evaluate differences between control and experimental conditions.

### Example 1

### Artificially Intellegence (AI)-Aided Virtual Screening for OTUD7B Small Molecule Inhibitors

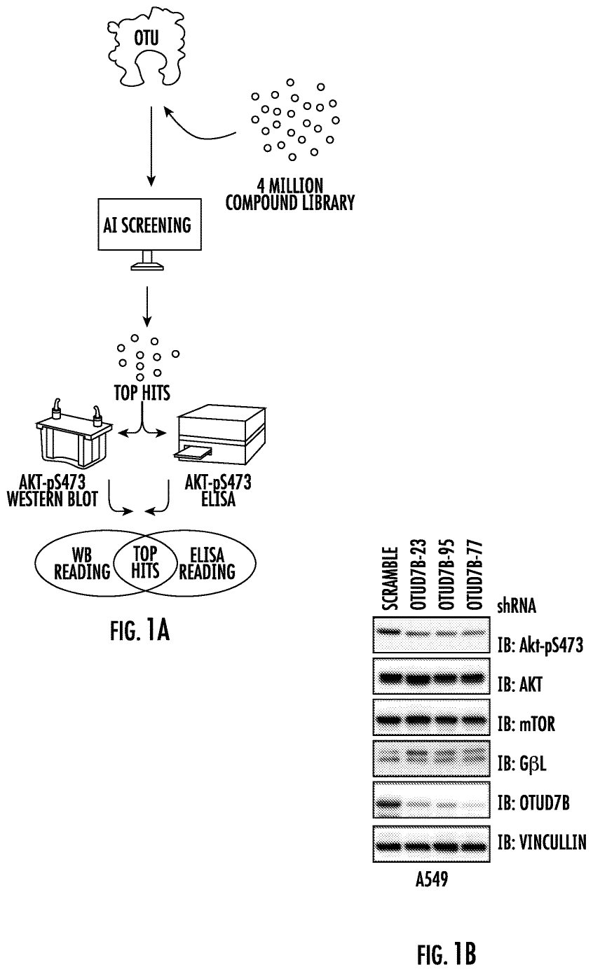

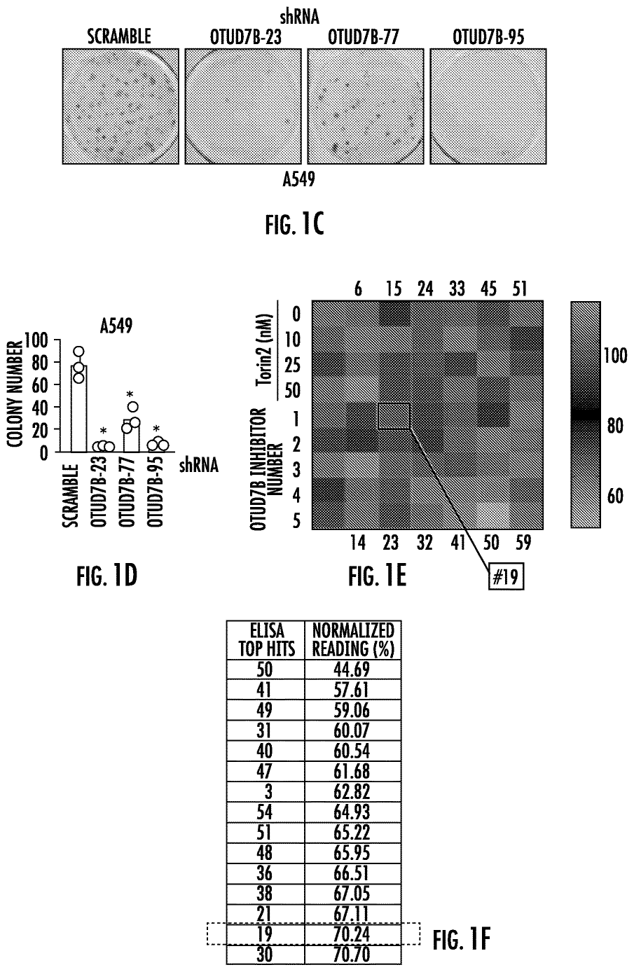

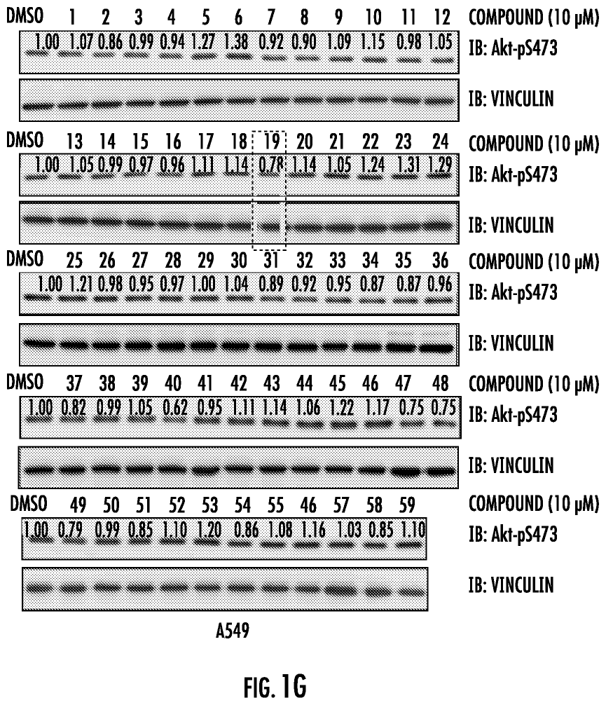

Previously, we reported OTUD7A as a vulnerability in Ewing sarcoma by deubiquitinating and stabilizing EWS-FL11, and we have successfully utilized the AtomNet model [37] to identify 7Ai as an OTUD7A catalytic inhibitor [35]. Relying on a solved crystal structure of the OTUD7B catalytic OTU domain (PDB: 5LRW), we tested 4 million commercially available, drug-like compounds in fitting into this structure in a virtual screen and purchased and tested a diverse set of 59 compounds from the top-scoring virtual hits (FIG. 1A).

### Example 2

### Validation and Selection of Viable OTUD7B Inhibitors in Cells

Considering previously genetic murine models and cell line-based studies firmly demonstrated that genetic OTUD7B depletion attenuated NSCLC (non-small cell lung cancer) tumor progression largely through inhibiting the OTUD7B/GL/Akt-pS473 signaling [19], we used NSCLC cells to examine effects of these 59 synthesized small molecules in suppressing Akt-pS473 signals. We first validated that in the NSCLC A549 cells, depletion of endogenous OTUD7B reduced Akt-pS473 (FIG. 1B), leading to subsequently reduced A549 cell growth in vitro (FIG. 1C-1D). Next, we used two approaches to search for possible OTUD7B inhibitors by monitoring Akt-pS473 changes including Akt-pS473 ELISA and western blotting in A549 cells treated with each of these 59 small molecules for 16 hrs at a concentration of 10 uM (FIG. 1A). In Akt-pS473 ELISA assays using Torin 2 (a pan-mTOR inhibitor) as a positive control and DMSO as a negative control (FIG. 1E), we found 15 compounds with an inhibitory ability above 30% (FIG. 1F). From the Akt-pS473 western blotting approach, we found 13 compounds suppressed phosphorylation by more than 10% (FIG. 1G-1H). Overlaying hits from both of these in-cell validation approaches led to identification of 10 common hits (FIG. 1I).

### Example 3

### Validation of OTUD7B Inhibitors in Multiple NSCLC Cell Lines

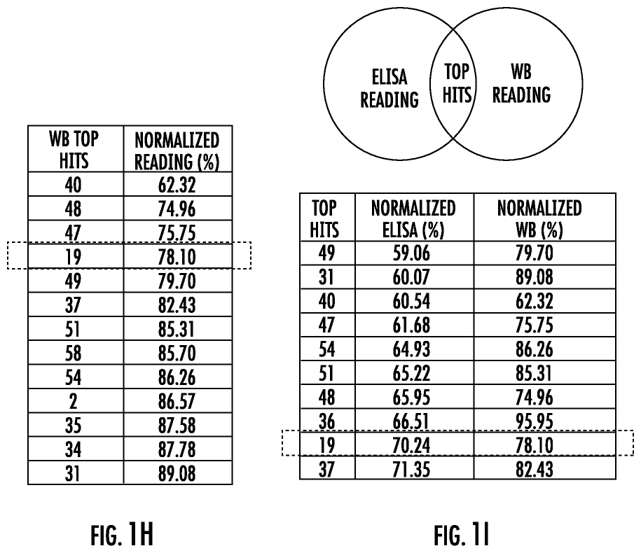

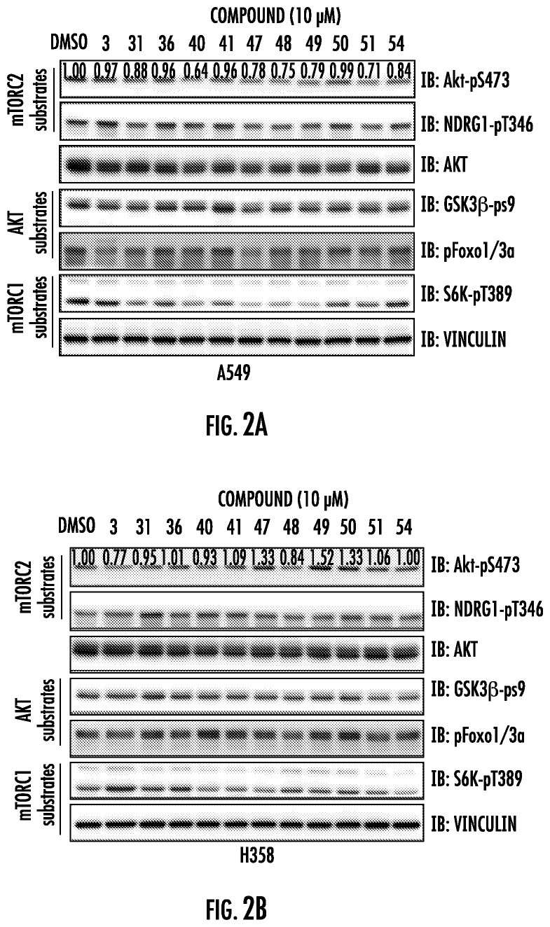

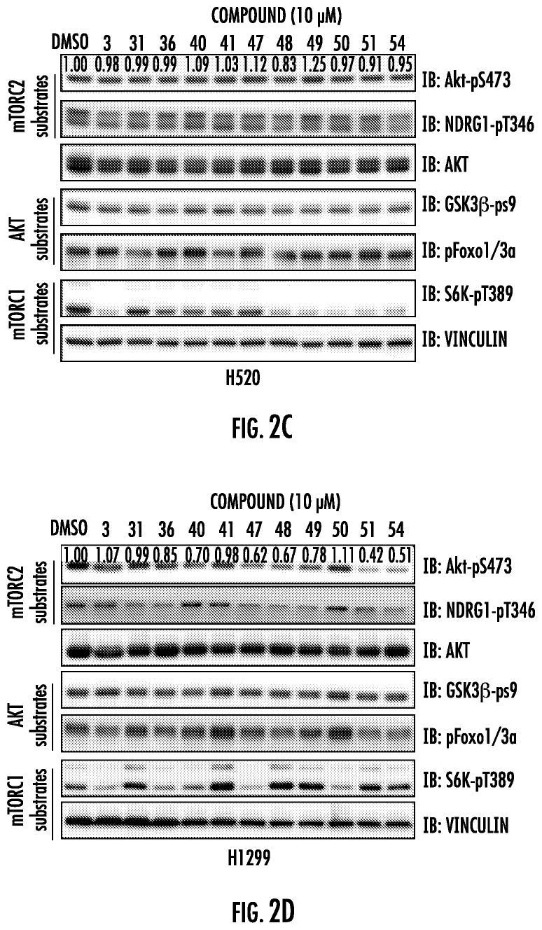

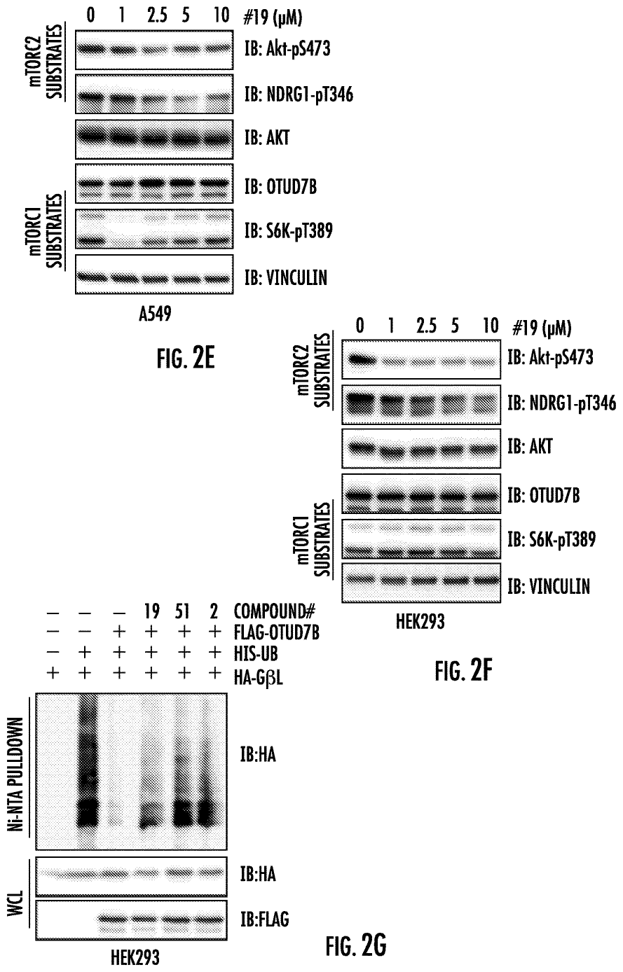

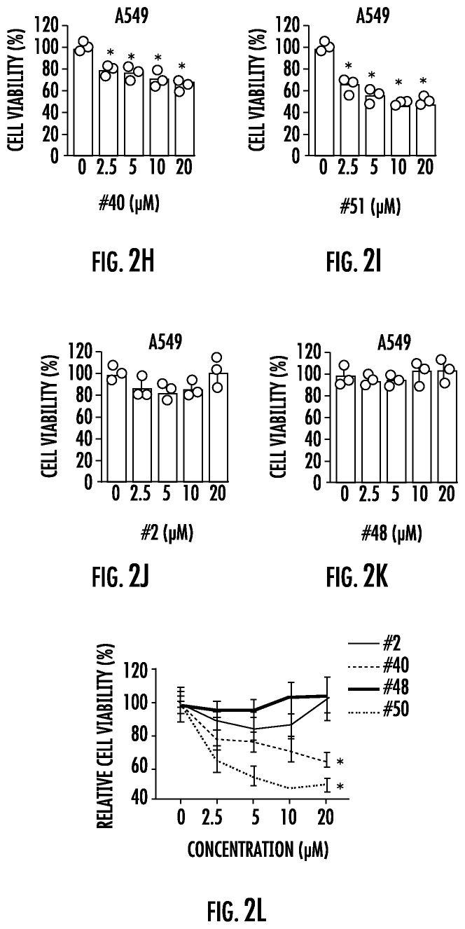

Considering the poised genetic composition from a single cell line may cause biased selection of OTUD7B inhibitors, as well as regulation of Akt-pS473 through OTUD7B-independent mechanisms, we further tested effects of 10 common hits identified from both Akt-pS473 ELISA and western blotting (FIG. 1I) in additional NSCLC cell lines, including A549 (FIG. 2A and FIG. S1A), H358 (FIG. 2B), H520 (FIG. 2C and FIG. S1B) and H1299 (FIG. 2D and FIG. S1C) at a single dose of 10 uM for 16 hrs. Among them, compound #2 (FIG. S1A-S1E), #40 (FIG. 2A-2D and FIG. S1F), #51 (FIG. 2A-2D) and #19 (FIG. 2E and FIG. S1G-S1I) showed effects in suppressing Akt-pS473 signal in at least 3 out of 4 NSCLC lines tested in both compound dose—(FIG. S1D, S1F, S1G, S1H, S1I) and time—(FIG. S1E) dependent manners. In addition, the compound #19 also efficiently suppressed Akt-pS473 in HEK293 cells (FIG. 2F), a cell line commonly used in studying regulation of mTOR signaling [38].

To further reveal if OTUD7B inhibitor-mediated suppression of Akt-pS473 is largely due to changes associated with OTUD7B-governed GβL ubiquitination, we examined effects of these compounds in modulating GβL ubiquitination in in-cell ubiquitination assays, and found that compounds #19, #52 and #2 partially blocked OTUD7B-induced GβL deubiquitination (FIG. 2G). Although these compounds reduced Akt-pS473 (which earmarks Akt activation to promote cell proliferation [20]), we found unlike compound #40 and #51 that suppressed A549 cell proliferation (FIG. 2H-2I), compound #2 and #48 failed to do so (FIG. 2J-2L). These data suggests that in addition to regulating OTUD7B/GβL/Akt-pS473 signaling, compounds #2 and #48 may also exert OTUD7B-independent effects in regulating cell proliferation, thus they were removed from our hit list but only serve as additional negative controls.

### Example 4

### Validation of OTUD7B Inhibitors Through In Vitro Deubiquitination Assays

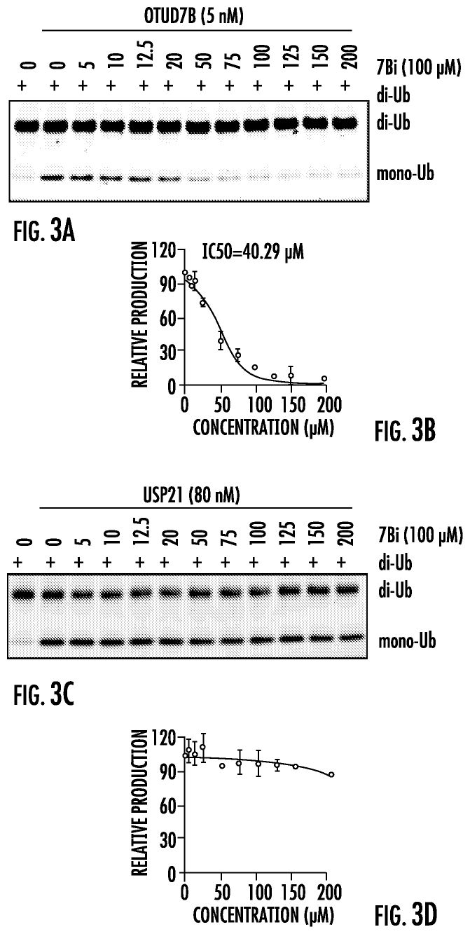

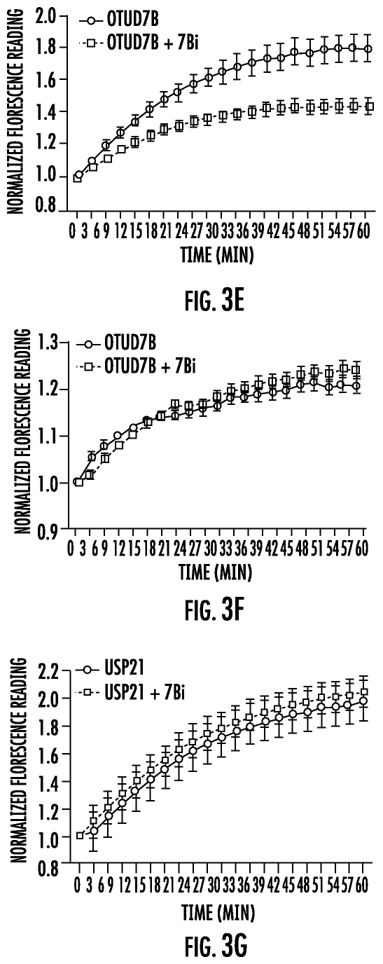

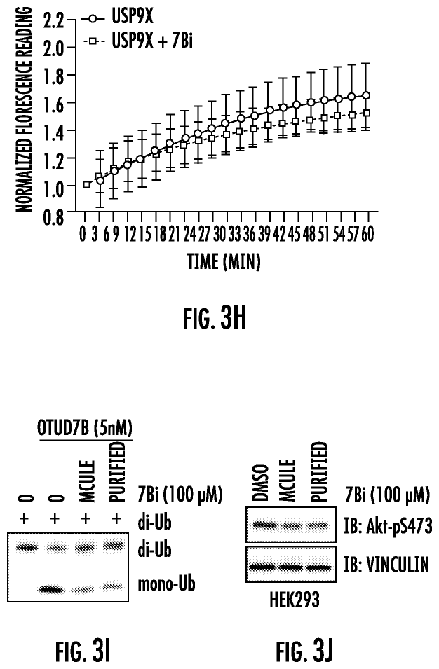

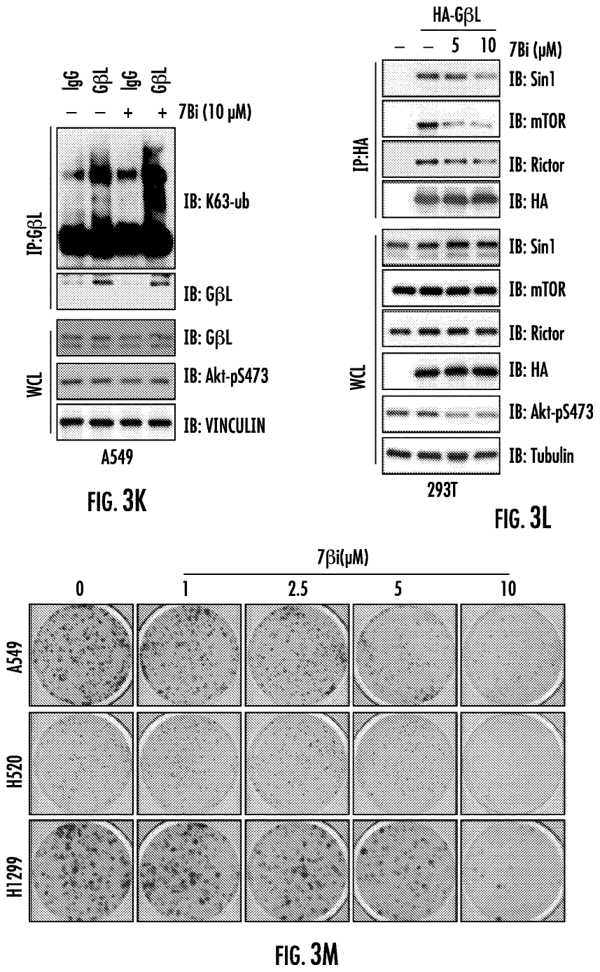

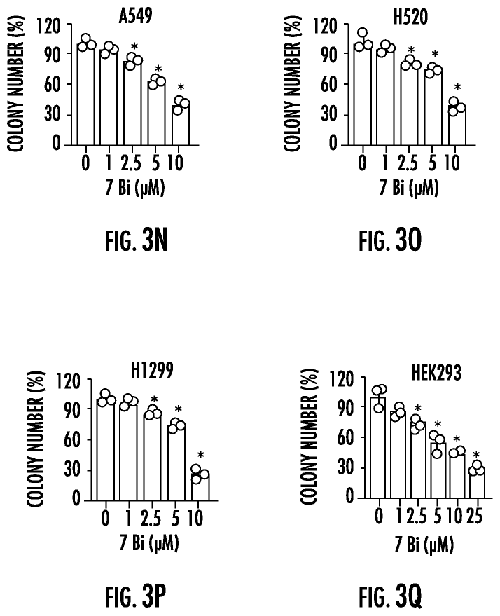

To provide direct evidence for inhibitory effects by these compounds on OTUD7B enzyme activity, we performed OTUD7B in vitro deubiquitinase assays using K11-linked di-ub as a substrate as previously reported [21]. In this assay, compounds #2, #40, #48 and #51 failed to inhibit the bacterially-purified OTUD7B enzyme in catalyzing the cleavage of K11-linked di-ub into mono-ub in vitro (FIG. S2A). On the other hand, compound #19 suppressed OTUD7B catalytic activity in this in vitro assay with an IC50 of ~40 μM (under the conditions of 0.5 uM K11-diub and 5 nM active OTUD7B) (FIG. 3A-3B). In addition, distinct from suppressing OTUD7B activity in vitro (FIG. S2B), compound #19 failed to inhibit other tested deubiquitinases including USP21 (FIG. 3C-3D), and USP9X (FIG. S2C), suggesting compound #19 is not a general deubiquitinase inhibitor. Given compound #19 suppresses both OTUD7B-mediated GβL deubiquitination in cells (FIG. 2G) and OTUD7B-mediated cleavage of K11-linked di-ub in vitro (FIG. 3A), we termed this compound as 7Bi to indicate its potential as a OTUD7B inhibitor. We further confirmed the specificity of 7Bi towards inhibiting OTUD7B by in vitro deubiquitination assays using ubiquitin-AMC as a substrate (without ubiquitin linkage effects), where we observed that 7Bi efficiently inhibited hydrolysis of ubiquitin-AMC in vitro by OTUD7B (FIG. 3E), but not its close family member OTUD7A (FIG. 3F), nor USP21 (FIG. 3G) or USP9X (FIG. 3H). To confirm the catalytic inhibitory activity of 7Bi is derived from its hypothesized structure but not contaminants from chemical synthesis, we purified commercially synthesized 7Bi (from 90% purity) by HPLC to obtain a purity of ~99% (FIG. S2D). Compared with commercially synthesized 7Bi with a 90% purity, HPLC purified 7Bi retained its ability to suppress OTUD7B-mediated K11-diub cleavage in vitro (FIG. 3I) and 7Bi-governed downregulation of Akt-pS473 signals in HEK293 cells (FIG. 3J).

### Example 5

7Bi Treatment Increases GβL Ubiquitination in Cells and Reduces NSCLC Cell Growth

To further examine effects of 7Bi in cells, we first observed that 7Bi treatment in A549 cells increased K63-linked ubiquitination of GβL at endogenous levels (FIG. 3K) presumably through inhibiting OTUD7B. This led to attenuated GβL binding with mTORC2 components including Sin1, Rictor and mTOR (FIG. 3L), which is consistent with a critical role of K63-linked GβL ubiquitination in facilitating the mTORC2 kinase complex formation [19]. Consistent with a role of Akt activation in facilitating cell proliferation and survival [20], 7Bi treatment reduced growth of multiple NSCLC cells including A549 (FIG. 3M-3N and FIG. S2E), H520 (FIG. 3O and FIG. S2F) and H1299 (FIG. 3P and FIG. S2G), as well as HEK293 cells (FIG. 3Q). Furthermore, 7Bi did not significantly affect OTUD7B protein levels in A549 and HEK293 cells (FIG. 2E-2F), nor significantly affected OTUD7B binding to GβL (FIG. S2H). Together, these data suggest that 7Bi (chemical structure shown in FIG. S2I) inhibits OTUD7B deubiquitinase activity in cells and in vitro to suppress cell proliferation.

Due to the size and side-chain flexibility within the targeted protein-protein interaction site, it is challenging to select a definitive binding pose for 7Bi from the ones suggested by the AtomNet screening platform. However, some general observations can be made: In the ubiquitin-bound crystal structure that served as a template for our screen, the C-terminal VLRLRG sequence (residues 70-75) of the ubiquitin ligand makes several lipophilic as well as charge-charge interactions with the binding site (FIG. S2J; light blue, thin sticks). In several of the poses for 7Bi, lipophilic cyclopentyl or methyl groups make similar lipophilic interactions with the surface of the binding site. Likewise, the amide bonds connecting the three central pyrazole rings may mimic the peptide back-bone of the ubiquitin. While 7Bi lacks the charged interaction of the endogenous ligand, it can engage in more direct contact with the small sub-pocket near F391. To illustrate the above discussion, an example pose for 7Bi is shown in FIG. S2J (yellow, bold sticks).

### Example 6

### Genetic and Pharmacological OTUD7B Inactivation Suppresses Leukemia Cell Proliferation

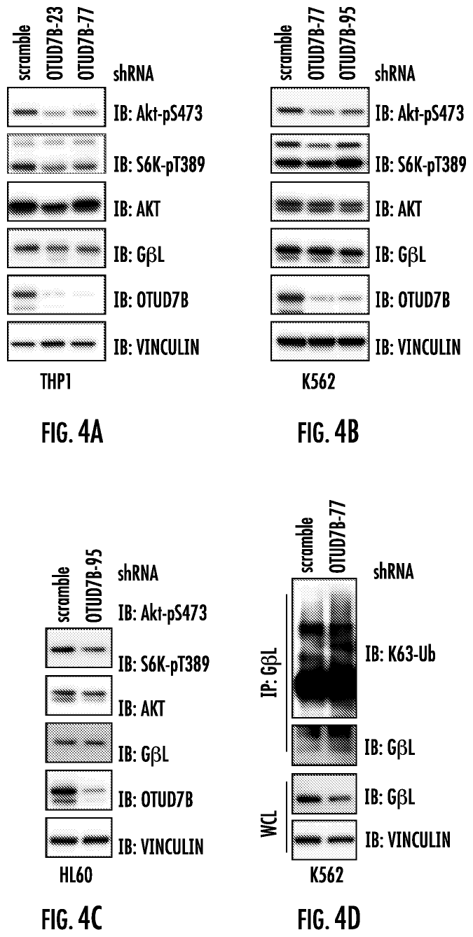

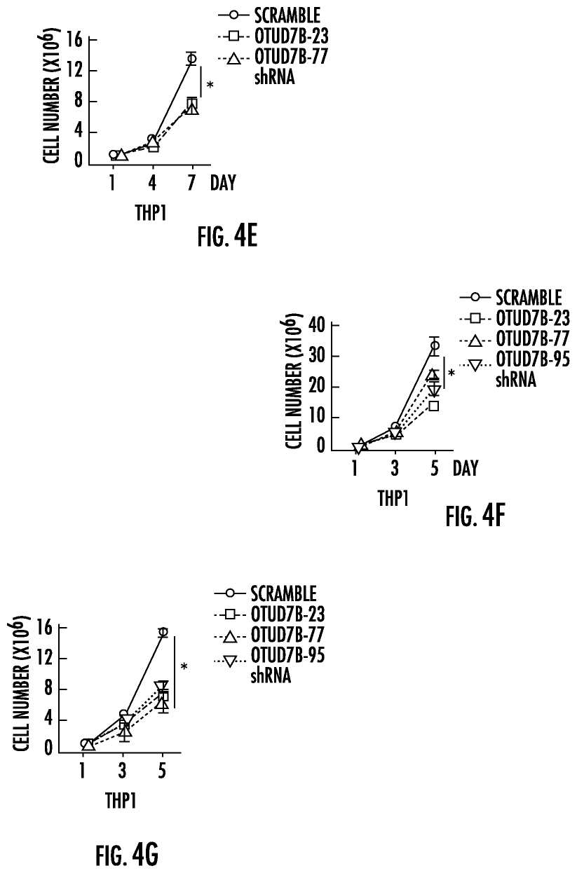

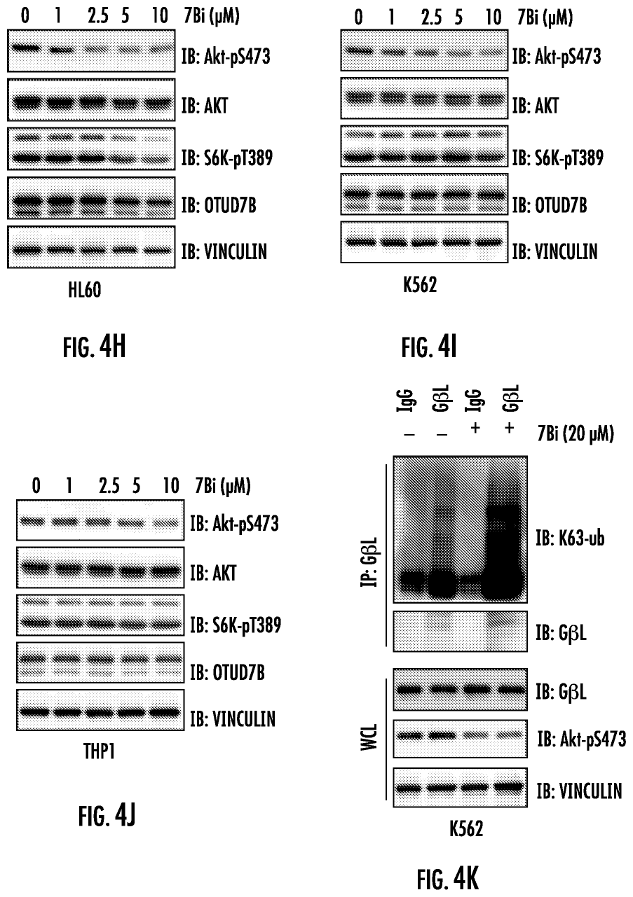

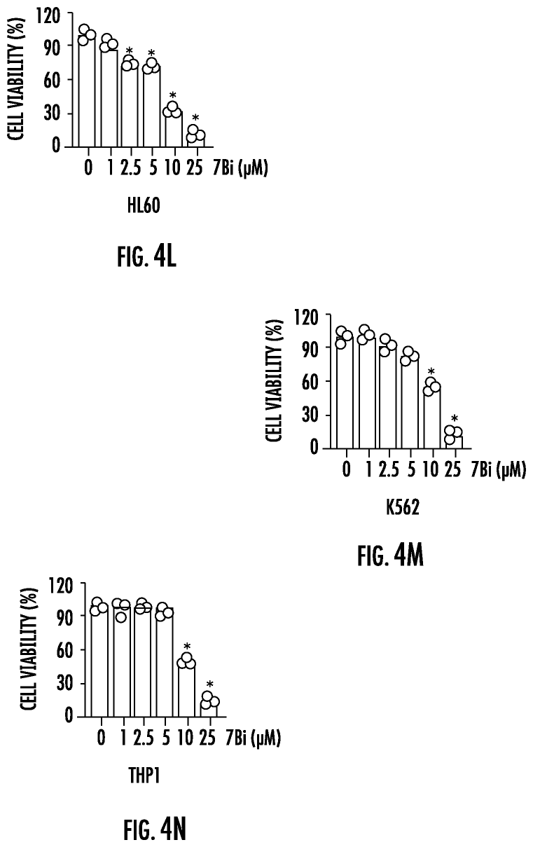

In addition to NSCLC, overexpression of OTUD7B was also observed in diffuse large B-cell lymphoma [30] and served as a prognostic marker. This promoted us to examine roles of OTUD7B in leukemia cells. We firstly depleted OTUD7B in THP1 (monocytes), HL-60 (acute promyelocytic leukemia) and K562 (chronic myelogenous leukemia) cells and found OTUD7B knockdown led to reduced Akt-pS473 signals (FIG. 4A-4C). Similar to NSCLC cells, depletion of OTUD7B in K562 cells also led to increased K63-linked GβL ubiquitination (FIG. 4D). Consistent with a reduction in Akt-pS473 signals, OTUD7B depletion reduced the growth of THP1 (FIG. 4E), K562 (FIG. 4F) and HL60 (FIG. 4G) cells in vitro. More importantly, pharmacological inhibition of OTUD7B by 7Bi reduced Akt-pS473 signals in HL60 (FIG. 4H), K562 (FIG. 4I) and THP1 (FIG. 4J) cells, mimicking effects of OTUD7B depletion. 7Bi treatment increased K63-linked GβL ubiquitination at endogenous levels in K562 cells (FIG. 4K), further supporting OTUD7B may govern leukemia cell proliferation through a similar OTUD7B/GβL/Akt signaling axis as in NSCLC. Consistent with an oncogenic role of OTUD7B in facilitating leukemia cell proliferation, inhibiting OTUD7B by 7Bi reduced proliferation of HL60 (FIG. 4L), K562 (FIG. 4M) and THP1 (FIG. 4N) cells. Together, these data not only support a critical role of OTUD7B in governing leukemia cell proliferation, but also confirm 7Bi as a potential OTUD7B small molecule inhibitor in suppressing leukemia cell proliferation.

### Discussion of Examples 1-6

The oncogenic feature of the deubiquitinase OTUD7B has been reported in NSCLC [19]. In this study, we use the AtomNet screening platform to perform an AI-aided virtual compound screening with a chemical library containing 4 million of structurally distinctive chemical compounds. Top 59 compounds were purchased and potential OTUD7B inhibitors were further screened by both Akt-pS473 ELISA and western blotting analyses in a NSCLC A549 cells. Overlaying common hits from both screens narrowed down candidates to 10 hits, and among them, 2 compounds (#19 and #51) survived after another two tiers of selection, including (1) exerting an ability to reduce Akt-pS473 in at least 3 out of 4 NSCLC cells; and (2) suppressing NSCLC cell growth in vitro. Both compounds were able to block OTUD7B-mediated GβL deubiquitination in cells. However, in in vitro OTUD7B deubiquitnation assays using K11-linked diUb as a substrate, only one compound #19—we termed 7Bi—showed an inhibitory effect, suggesting the other two compounds likely indirectly regulate GβL-ubiquitination and Akt-pS473 signals in cells. We further confirmed that 7Bi treatment efficiently reduced growth of multiple NSCLC cells and HEK293 cells. We further found OTUD7B also governed various leukemia cell proliferation and 7Bi treatment also efficiently reduced leukemia cell proliferation in vitro. Together, our study identifies a small molecule 7Bi as a possible OTUD7B catalytic inhibitor, which may be a viable therapeutic agent in treating cancers with OTUD7B overexpression including breast cancer [28], lung cancer [29], diffuse large B-cell lymphoma [30] and NSCLC [31], as well as other human disorders including myocardial infarction [32]. To the best of our knowledge, this is the first non-covalent small molecule inhibitor of OTUD7B.

Notably, the IC50 values for 7Bi to inhibit Akt-pS473 signals in NSCLC cells range from 1-10 uM and the IC50 values for 7Bi to suppress NSCLC cell growth are about 2.5 uM, thus further in-depth SAR (structure-activity relationship) studies are necessary to improve its potency. In addition, the IC50 for #19 in this in vitro OTUD7B deubiquitination assays is determined by the amounts of di-ub (substrate) and OTUD7B, as well as the reaction time, thus it does not provide a comparable evaluation to its in-cell IC50 values. In addition, although 7Bi does not inhibit OTUD7A, USP21 and USP9X in vitro, the specificity of 7Bi over a broader range of DUBs remains to be further determined. Our current in-cell and in vitro evaluations suggest 7Bi as a potential OTUD7B inhibitor while the target engagement has not been tested and further in-depth analyses are required. Moreover, effects of 7Bi on tumor growth in animal models, as well as the bioavailability and pharmacological properties of 7Bi also need to be profiled and examined. Nonetheless, identification of 7Bi as the first small molecule inhibitor of OTUD7B is a start for the development of viable therapeutic agents targeting OTUD7B. We have searched Scifinder for literature or patents on 7Bi, and there were none. We also found no reported bioactivities for closely related compounds. In addition, the AtomNet technology seems to be able to find distinct sets of compound inhibitors for closely related deubiquitinase members such as OTUD7A [35] and OTUD7B (this study). Cross-activity examinations confirmed that 7Ai does not significantly suppress OTUD7B activity in cell [35] and 7Bi does not inhibitor OTUD7A activity in vitro (FIG. 3F). However, due to the nature of the AtomNet technology, it is difficult to generate high quality simulation structures to reveal reliable binding pose of 7Bi that can guide mutagenesis studies for further validation. This will require solving the structure of 7Bi bound to OTUD7B.

Given that OTUD7B exerts both deubiquitinase dependent- and independent-functions in tumorigenesis and immune regulation, identification of OTUD7B catalytic inhibitors or protein-protein interaction inhibitors may lead to further development of OTUD7B-PROTACs or DUBTACs by using discovered OTUD7B binding chemical structures, which could further enhance OTUD7B targeted therapeutic efficacy and broaden its potential applications in disease treatment. Notably, considering the immune regulatory function of OTUD7B that OTUD7B may facilitate T cell activation [26], effects of OTUD7B inhibitors in possibly creating an immune suppressive environment warrants further in-depth investigations and if so cautions should be taken when OTUD7B inhibition is used in clinic.

### Summary of Examples

In this study aided by the AtomNet® technology, we screened 4 million compounds and identified a potential OTUD7B catalytic inhibitor that we term as 7Bi. 7Bi treatment in NSCLC and HEK293 cells efficiently reduced Akt-pS473 signals by interfering with OTUD7B-directed GβL deubiquitination, leading to reduced cell growth. In addition, we report an oncogenic function of OTUD7B in leukemia by similarly controlling K63-linked GβL ubiquitination and mTORC2 activity in governing leukemia cell proliferation. We further demonstrate that 7Bi also efficiently suppresses leukemia cell proliferation. Together, our study provides the chemical structure for a possible OTUD7B inhibitor that can be further improved by medicinal chemistry or PROTAC/DUBTAC approaches, with potentials as viable therapeutic agents in treating cancer with OTUD7B overexpression. Additional studies to determine the binding affinity and structural insights would be helpful for next rounds of SAR studies that aim to improve the potency and specificity of 7Bi. Further PK/PD studies to examine and improve the chemical properties of 7Bi, and more importantly, examination of effects of 7Bi or improved 7Bi in suppressing OTUD7B activity and oncogenic function in murine cancer models would be needed to gain insights for its therapeutic potential in pre-clinical models.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. Deng L, Meng T, Chen L, Wei W, and Wang P, The role of ubiquitination in tumorigenesis and targeted drug discovery. Signal Transduct Target Ther, 5(1): p. 11.2020.
2. Lange S M, Armstrong L A, and Kulathu Y, Deubiquitinases: From mechanisms to their inhibition by small molecules. Mol Cell, 82(1): p. 15-29.2022.
3. Mevissen T E T and Komander D, Mechanisms of Deubiquitinase Specificity and Regulation. Annu Rev Biochem, 86: p. 159-192.2017.
4. Kwasna D, Abdul Rehman S A, Natarajan J, Matthews S, Madden R, De Cesare V, Weidlich S, Virdee S, Ahel I, Gibbs-Seymour I, and Kulathu Y, Discovery and Characterization of ZUFSP/ZUP1, a Distinct Deubiquitinase Class Important for Genome Stability. Mol Cell, 70(1): p. 150-164 e6.2018.
5. Bonacci T and Emanuele M J, Dissenting degradation: Deubiquitinases in cell cycle and cancer. Semin Cancer Biol, 67(Pt 2): p. 145-158.2020.
6. Zhu D, Xu R, Huang X, Tang Z, Tian Y, Zhang J, and Zheng X, Deubiquitinating enzyme OTUB1 promotes cancer cell immunosuppression via preventing ER-associated degradation of immune checkpoint protein PD-L1. Cell Death Differ, 28(6): p. 1773-1789.2021.
7. Baietti M F, Simicek M, Abbasi Asbagh L, Radaelli E, Lievens S, Crowther J, Steklov M, Aushev V N, Martinez Garcia D, Tavernier J, and Sablina A A, OTUB1 triggers lung cancer development by inhibiting RAS monoubiquitination. EMBO Mol Med, 8(3): p. 288-303.2016.
8. Iglesias-Gato D, Chuan Y C, Jiang N, Svensson C, Bao J, Paul I, Egevad L, Kessler B M, Wikstrom P, Niu Y, and Flores-Morales A, OTUB1 deubiquitinating enzyme promotes prostate cancer cell invasion in vitro and tumorigenesis in vivo. Mol Cancer, 14: p. 8.2015.
9. Zhang H H, Li C, Ren J W, Liu L, Du X H, Gao J, Liu T, and Li S Z, OTUB1 facilitates bladder cancer progression by stabilizing ATF6 in response to endoplasmic reticulum stress. Cancer Sci, 112(6): p. 2199-2209.2021.
10. Zhou K, Mai H, Zheng S, Cai W, Yang X, Chen Z, and Zhan B, OTUB1-mediated deubiquitination of FOXM1 up-regulates ECT-2 to promote tumor progression in renal cell carcinoma. Cell Biosci, 10: p. 50.2020.
11. Liu T, Jiang L, Tavana 0, and Gu W, The Deubiquitylase OTUB1 Mediates Ferroptosis via Stabilization of SLC7A11. Cancer Res, 79(8): p. 1913-1924.2019.
12. Moree S E, Maneix L, Iakova P, Stossi F, Sahin E, and Catic A, Imaging-Based Screening of Deubiquitinating Proteases Identifies Otubain-1 as a Stabilizer of c-MYC. Cancers (Basel), 14(3).2022.
13. Yuan L, Lv Y, Li H, Gao H, Song S, Zhang Y, Xing G, Kong X, Wang L, Li Y, Zhou T, Gao D, Xiao Z X, Yin Y, Wei W, He F, and Zhang L, Deubiquitylase OTUD3 regulates PTEN stability and suppresses tumorigenesis. Nat Cell Biol, 17(9): p. 1169-81.2015.
14. Pu Q, Lv Y R, Dong K, Geng W W, and Gao H D, Tumor suppressor OTUD3 induces growth inhibition and apoptosis by directly deubiquitinating and stabilizing p53 in invasive breast carcinoma cells. BMC Cancer, 20(1): p. 583.2020.
15. Du T, Li H, Fan Y, Yuan L, Guo X, Zhu Q, Yao Y, Li X, Liu C, Yu X, Liu Z, Cui C P, Han C, and Zhang L, The deubiquitylase OTUD3 stabilizes GRP78 and promotes lung tumorigenesis. Nat Commun, 10(1): p. 2914.2019.
16. Zhang Z, Fang X, Wu X, Ling L, Chu F, Li J, Wang S, Zang J, Zhang B, Ye S, Zhang L, Yang B, Lin S, Huang H, Wang A, and Zhou F, Acetylation-Dependent Deubiquitinase OTUD3 Controls MAVS Activation in Innate Antiviral Immunity. Mol Cell, 79(2): p. 304-319 e7.2020.
17. Cai X, Zhou Z, Zhu J, Liu X, Ouyang G, Wang J, Li Z, Li X, Zha H, Zhu C, Rong F, Tang J, Liao Q, Chen X, and Xiao W, Opposing effects of deubiquitinase OTUD3 in innate immunity against RNA and DNA viruses. Cell Rep, 39(10): p. 110920.2022.
18. Zhou N, Qi H, Liu J, Zhang G, Liu J, Liu N, Zhu M, Zhao X, Song C, Zhou Z, Gong J, Li R, Bai X, Jin Y, Song Y, and Yin Y, Deubiquitinase OTUD3 regulates metabolism homeostasis in response to nutritional stresses. Cell Metab, 34(7): p. 1023-1041 e8.2022.
19. Wang B, Jie Z, Joo D, Ordureau A, Liu P, Gan W, Guo J, Zhang J, North B J, Dai X, Cheng X, Bian X, Zhang L, Harper J W, Sun S C, and Wei W, TRAF2 and OTUD7B govern a ubiquitin-dependent switch that regulates mTORC2 signalling. Nature, 545(7654): p. 365-369.2017.
20. Manning B D and Toker A, AKT/PKB Signaling: Navigating the Network. Cell, 169(3): p. 381-405.2017.
21. Bonacci T, Suzuki A, Grant G D, Stanley N, Cook J G, Brown N G, and Emanuele M J, Cezanne/OTUD7B is a cell cycle-regulated deubiquitinase that antagonizes the degradation of APC/C substrates. EMBO J, 37(16).2018.
22. Gong Z, Li A, Ding J, Li Q, Zhang L, Li Y, Meng Z, Chen F, Huang J, Zhou D, Hu R, Ye J, Liu W, and You H, OTUD7B Deubiquitinates LSD1 to Govern Its Binding Partner Specificity, Homeostasis, and Breast Cancer Metastasis. Adv Sci (Weinh), 8(15): p. e2004504.2021.
23. Tang J, Wu Z, Tian Z, Chen W, and Wu G, OTUD7B stabilizes estrogen receptor alpha and promotes breast cancer cell proliferation. Cell Death Dis, 12(6): p. 534.2021.
24. Bremm A, Moniz S, Mader J, Rocha S, and Komander D, Cezanne (OTUD7B) regulates HIF-1alpha homeostasis in a proteasome-independent manner. EMBO Rep, 15(12): p. 1268-77.2014.
25. Hu H, Brittain G C, Chang J H, Puebla-Osorio N, Jin J, Zal A, Xiao Y, Cheng X, Chang M, Fu Y X, Zal T, Zhu C, and Sun S C, OTUD7B controls non-canonical NF-kappaB activation through deubiquitination of TRAF3. Nature, 494(7437): p. 371-4.2013.
26. Hu H, Wang H, Xiao Y, Jin J, Chang J H, Zou Q, Xie X, Cheng X, and Sun S C, Otud7b facilitates T cell activation and inflammatory responses by regulating Zap70 ubiquitination. J Exp Med, 213(3): p. 399-414.2016.
27. Xie W, Tian S, Yang J, Cai S, Jin S, Zhou T, Wu Y, Chen Z, Ji Y, and Cui J, OTUD7B deubiquitinates SQSTM1/p62 and promotes IRF3 degradation to regulate antiviral immunity. Autophagy, 18(10): p. 2288-2302.2022.
28. Chiu H W, Lin H Y, Tseng I J, and Lin Y F, OTUD7B upregulation predicts a poor response to paclitaxel in patients with triple-negative breast cancer. Oncotarget, 9(1): p. 553-565.2018.
29. Lin D D, Shen Y, Qiao S, Liu W W, Zheng L, Wang Y N, Cui N, Wang Y F, Zhao S, and Shi J H, Upregulation of OTUD7B (Cezanne) Promotes Tumor Progression via AKT/VEGF Pathway in Lung Squamous Carcinoma and Adenocarcinoma. Front Oncol, 9: p. 862.2019.
30. Qiu S, Liu Y, Gui A, Xia Z, Liu W, Gu J J, Zuo J, Yang L, and Zhang Q, Deubiquitinase OTUD7B is a potential prognostic biomarker in diffuse large B-cell lymphoma. J Cancer, 13(3): p. 998-1004.2022.
31. Zhang B, Wang H, Yang L, Zhang Y, Wang P, Huang G, Zheng J, Ren H, and Qin S, OTUD7B and NIK expression in non-small cell lung cancer: Association with clinicopathological features and prognostic implications. Pathol Res Pract, 212(10): p. 893-898.2016.
32. Zhang J, Zha Y, Jiao Y, Li Y, Wang J, and Zhang S, OTUD7B (Cezanne) ameliorates fibrosis after myocardial infarction via FAK-ERK/P38 MAPK signaling pathway. Arch Biochem Biophys, 724: p. 109266.2022.
33. Zhu Z, Zhou X, Du H, Cloer E W, Zhang J, Mei L, Wang Y, Tan X, Hepperla A J, Simon J M, Cook J G, Major M B, Dotti G, and Liu P, STING Suppresses Mitochondrial VDAC2 to Govern RCC Growth Independent of Innate Immunity. Adv Sci (Weinh): p. e2203718.2022.
34. Mevissen T E, Hospenthal M K, Geurink P P, Elliott P R, Akutsu M, Arnaudo N, Ekkebus R, Kulathu Y, Wauer T, El Oualid F, Freund S M, Ovaa H, and Komander D, OTU deubiquitinases reveal mechanisms of linkage specificity and enable ubiquitin chain restriction analysis. Cell, 154(1): p. 169-84.2013.
35. Su S, Chen J, Jiang Y, Wang Y, Vital T, Zhang J, Laggner C, Nguyen K T, Zhu Z, Prevatte A W, Barker N K, Herring L E, Davis I J, and Liu P, SPOP and OTUD7A Control EWS-FLI1 Protein Stability to Govern Ewing Sarcoma Growth. Adv Sci (Weinh), 8(14): p. e2004846.2021.
36. Mevissen T E T, Kulathu Y, Mulder M P C, Geurink P P, Maslen S L, Gersch M, Elliott P R, Burke J E, van Tol B D M, Akutsu M, Oualid F E, Kawasaki M, Freund S M V, Ovaa H, and Komander D, Molecular basis of Lys11-polyubiquitin specificity in the deubiquitinase Cezanne. Nature, 538(7625): p. 402-405.2016.
37. Izhar Wallach M D, Abraham Heifets, AtomNet: A Deep Convolutional Neural Network for Bioactivity Prediction in Structure-based Drug Discovery. arXiv: p. 1510.02855 2015.
38. Kim D H, Sarbassov D D, Ali S M, King J E, Latek R R, Erdjument-Bromage H, Tempst P, and Sabatini D M, mTOR interacts with raptor to form a nutrient-sensitive complex that signals to the cell growth machinery. Cell, 110(2): p. 163-75.2002.

It will be understood that various details of the presently disclosed subject matter may be changed without departing from the scope of the presently disclosed subject matter. Furthermore, the foregoing description is for the purpose of illustration only, and not for the purpose of limitation.

1. A composition for targeting OTUD7B, the composition comprising a component sufficient to block and/or reduce OTUD7B-mediated deubiquitination of GβL in a cell, wherein the component comprises 7Bi and variants thereof.

2. The composition of claim 1, wherein the component is a catalytic inhibitor, wherein the catalytic inhibitor inhibits the catalytic activity of OTUD7B.

3. The composition of claim 1, wherein blocking and/or reducing OTUD7B-mediated deubiquitination of GβL substantially inhibits, reduces and/or alters Akt-pS473 signaling in a cell, wherein the Akt-pS473 signaling in a cell is inhibited, reduced and/or altered by about 50% or more.

4. The composition of claim 1, wherein the cell is a human cell.

5. (canceled)

6. The composition of claim 1, wherein 7Bi comprises the following structure:

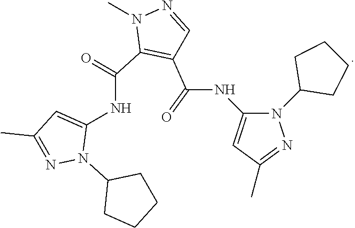 [SMILES: Cc1cc(NC(=O)c2cnn(C)c2C(=O)Nc2cc(C)nn2C2CCCC2)n(C2CCCC2)n1]

7. The composition of claim 1, wherein 7Bi is in the composition at a concentration sufficient to provide a dosage of about 10 μM when administered to a subject.

8. The composition of claim 1, wherein the composition comprises a delivery vehicle for the component, wherein the delivery vehicle is an expression vector, nanoparticle, liposome or vesicle.

9. The composition of claim 1, wherein the composition is configured for treating a cancer, wherein the cancer is breast cancer, lung cancer, or leukemia.

10. An OTUD7B catalytic inhibitor, the catalytic inhibitor comprising a 7Bi compound or variant thereof.

11. The catalytic inhibitor of claim 10, wherein the catalytic inhibitor is configured to substantially limit cancer cell growth in vivo by blocking and/or reducing OTUD7B-mediated deubiquitination of GβL in a cell.

12. The catalytic inhibitor of claim 10, wherein the 7Bi compound comprises the following structure:

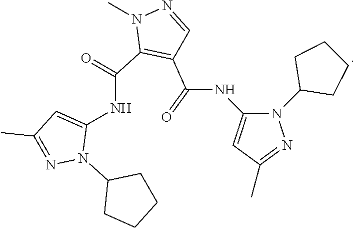 [SMILES: Cc1cc(NC(=O)c2cnn(C)c2C(=O)Nc2cc(C)nn2C2CCCC2)n(C2CCCC2)n1]

13. A method of treating cancer and related conditions, the method comprising administering to a subject having cancer or suspected of suffering from cancer a composition comprising a OTUD7B catalytic inhibitor, wherein the OTUD7B catalytic inhibitor comprises a 7Bi or variant thereof.

14. The method of claim 13, wherein the component blocks and or reduces OTUD7B-mediated deubiquitination of GβL in a cell in the subject.

15. The method of claim 13, wherein the component is a catalytic inhibitor, wherein the catalytic inhibitor inhibits the catalytic activity of OTUD7B.

16. The method of claim 13, wherein blocking and/or reducing OTUD7B-mediated deubiquitination of GβL substantially inhibits, reduces and/or alters Akt-pS473 signaling in a cell, wherein the Akt-pS473 signaling in a cell is inhibited, reduced and/or altered by about 50% or more.

17. The method of claim 13, wherein the 7Bi comprises the following structure:

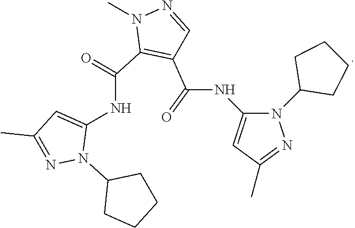 [SMILES: Cc1cc(NC(=O)c2cnn(C)c2C(=O)Nc2cc(C)nn2C2CCCC2)n(C2CCCC2)n1]

18. The method of claim 13, wherein the subject is suffering from a cancer or is believed to be suffering from a cancer, wherein the cancer is breast cancer, lung cancer, or leukemia.

19. The method of claim 13, wherein 7Bi is in the composition at a concentration sufficient to provide a dosage of about 10 μM when administered to a subject.

20. The method of claim 13, wherein the composition comprises a delivery vehicle for the component, wherein the delivery vehicle is an expression vector, nanoparticle, liposome or vesicle.

21. The method of claim 13 wherein the composition is co-administered to the subject with at least one chemotherapeutic drug.

22. (canceled)


SEQUENCE LISTING (3 sequences)

SEQ ID NO: 1 - DNA, 21 bp, synthetic construct
  misc_feature 1..21: note = shRNA sequence
  source 1..21: mol_type = other DNA
gcaaggaggctaaacaaagtt

SEQ ID NO: 2 - DNA, 21 bp, synthetic construct
  misc_feature 1..21: note = shRNA sequence
  source 1..21: mol_type = other DNA
caaagttaagctcaactaatt

SEQ ID NO: 3 - DNA, 21 bp, synthetic construct
  misc_feature 1..21: note = shRNA sequence
  source 1..21: mol_type = other DNA
tggaaatgctcacggtttata